(19) United States
(12) Patent Application Publication
(10) Pub. No.: US 2025/0185893 A1
(43) Pub. Date: Jun. 12, 2025

(54) SEALING DEVICE FOR ENDOSCOPE INTUBATION AND ENDOSCOPE

(71) Applicant: Micro-Tech (Nanjing) Co., Ltd., Nanjing (CN)

(72) Inventors: Wei Yang, Nanjing (CN); Zhenghua Shen, Nanjing (CN); Teng Zhang, Nanjing (CN)

(21) Appl. No.: 18/971,178

(22) Filed: Dec. 6, 2024

(30) Foreign Application Priority Data
Dec. 7, 2023 (CN) 2023233440721

(51) Int. Cl. A61B 1/00

(52) U.S. Cl. CPC A61B 1/00137; A61B 1/00002

(57) ABSTRACT

A sealing device for endoscope intubation and an endoscope are provided in the present disclosure, which includes: a main body part, wherein the main body part has an axis, and is used for connecting to the catheter, at least a portion of the main body part is configured to be capable of being inserted into the tracheal intubation, and there is a gap between the main body part and the inner tube wall of the tracheal intubation; a sealing part, which is annularly arranged on the main body part, wherein the sealing part is configured to seal the gap; and a friction part, which is arranged on the main body part, wherein at least portion of the friction part is configured to abut against the inner wall of the tracheal intubation.

### CROSS-REFERENCES TO RELATED APPLICATIONS

The present disclosure claims priority to Chinese Patent Application No. 2023233440721, entitled “SEALING DEVICE FOR ENDOSCOPE INTUBATION AND ENDOSCOPE” filed on Dec. 7, 2023 with the Chinese Patent Office, the entire contents of which are incorporated herein by reference.

### TECHNICAL FIELD

The present disclosure belongs to the technical field of endoscope, and particularly relates to a sealing device for endoscope intubation and an endoscope.

### BACKGROUND

At present, minimally invasive technology based on electronic endoscope clinically is widely used in many hospital departments such as respiratory department, digestive department, urology department and otolaryngology department, which is mainly used to screen organ lesions and carry out related interventions to carry out endoscopic surgery.

Among them, the respiratory intensive care unit conducts tracheal intubation under visual monitoring to guide the intubation to the appropriate position. Ventilation and oxygen supply are sometimes needed in the operation process, which requires that the joint of the catheter of the tracheoscope and the tracheal intubation has a certain sealing performance to avoid air leakage. At the same time, the tracheal intubation needs to move with the tracheoscope during the operation process, and be performed in a visual state during the whole operation to avoid damaging the airway.

### SUMMARY

The object of the present disclosure is that the example of the present disclosure provides a sealing device for endoscope intubation, aiming at solving the above technical problems; another object of the example of the present disclosure is to provide an endoscope.

Technical solution is as follows. A sealing device for endoscope intubation according to the embodiments of the present disclosure, which is configured for sealing connection of a catheter of the tracheoscope and a tracheal intubation, includes:
a main body part, wherein the main body part has an axis, the main body part is configured for connecting to the catheter, at least a portion of the main body part is configured to be capable of being inserted into the tracheal intubation, and there is a gap between the main body part and the inner tube wall of the tracheal intubation;
a sealing part, which is annularly arranged on the main body part, wherein the sealing part is configured to seal the gap;
a friction part, which is arranged on the main body part, wherein at least portion of the friction part is configured to abut against the inner wall of the tracheal intubation.

In some embodiments, the friction part is configured to generate an elastic deformation when the main body part is inserted into the tracheal intubation, to abut against the inner wall of the tracheal intubation.

In some embodiments, a plurality of friction parts are provided, and the plurality of friction parts are arranged at intervals in a circumferential direction around the axis.

In some embodiments, the extending direction of the friction part intersects or is parallel to the extending direction of the axis.

In some embodiments, the friction part extends spirally around the axis.

In some embodiments, the main body part has a distal end and a proximal end in the extending direction of the axis; and
the friction part has a first surface facing away from the main body part, and the vertical distance between at least part of the first surface and the axis gradually increases from the distal end to the proximal end.

In some embodiments, the maximum dimension of the main body part in the direction perpendicular to the axis gradually increases from the distal end to the proximal end; and
the distance between the first surface and the outer surface of the main body part gradually increases or is the same from the distal end to the proximal end.

In some embodiments, the maximum dimension of the main body part in the direction perpendicular to the axis is the same from the distal end to the proximal end; and
the distance between the first surface and the outer surface of the main body part gradually increases from the distal end to the proximal end.

In some embodiments, the main body part has a distal end and a proximal end in the extending direction of the axis; and
the sealing part is provided with a first guide surface, the first guide surface is arranged towards the distal end, and the first guide surface is configured for guiding the sealing part to be embedded into the gap.

In some embodiments, the main body part has a distal end and a proximal end in the extending direction of the axis; and
the friction part is provided with a second guide surface, the second guide surface is arranged towards the distal end and the second guide surface is configured for guiding the friction part to be embedded into the gap.

In some embodiments, a plurality of sealing parts are provided, and the plurality of sealing parts are arranged along the extending direction of the axis.

In some embodiments, the friction part is connected with the sealing part;
and/or the main body part has a distal end and a proximal end in the extending direction of the axis, and at least portion of the friction part is arranged at one side of the sealing part close to the distal end.

In some embodiments, the main body part, the sealing part and the friction part are integrally formed.

Correspondingly, an endoscope according to the embodiments of the present disclosure includes the sealing device for endoscope intubation as described above.

In some embodiments, the endoscope further includes: an operating handle and a catheter connected with the operating handle, wherein the sealing device for endoscope intubation is sleeved over the catheter, and the sealing device for endoscope intubation is fixedly connected with the operating handle.

Beneficial effects are as follows. The sealing device for endoscope intubation of an embodiment of the present disclosure is configured for sealing connection between a catheter of a tracheoscope and a tracheal intubation, including a main body part, a sealing part, and a friction part, wherein the main body part has an axis, the main body part is configured for connecting to the catheter, at least a portion of the main body part is configured to be able to be inserted into the tracheal intubation, and there is a gap between the main body part and the inner tube wall of the tracheal intubation; the sealing part is annularly arranged on the main body part, and the sealing part is configured to be able to seal the gap; the friction part is arranged on the main body part, and at least portion of the friction part is configured to abut against the inner wall of the tracheal intubation; and the sealing connection of the catheter and the tracheal intubation is realized by the main body part and the sealing part to avoid leakage when oxygen is delivered by the tracheal intubation, while the friction part abuts against the tracheal intubation in the gap to form a certain friction force, so as to maintain the synchronous mobility of the catheter and the tracheal intubation.

The endoscope of an embodiment of the present disclosure includes the above-mentioned sealing device for endoscope intubation, and it can be understood that the endoscope can have all the technical features and corresponding beneficial effects of the above-mentioned sealing device for endoscope intubation, which will not be described in detail here.

### BRIEF DESCRIPTION OF THE DRAWINGS

In order to more clearly illustrate technical solutions of the present disclosure, the drawings needed in the description of the examples will be briefly introduced below. It is obvious that the drawings in the following description are some examples of the present disclosure, for a person of ordinary skill in the art, other drawings can be obtained from these drawings without inventive efforts.

FIG. 1 is a schematic structural diagram of an operating handle, a catheter, and a sealing device for endoscope intubation of Example 1 of the present disclosure;

FIG. 2 is a schematic structural diagram of an operating handle, a catheter, and a sealing device for endoscope intubation connecting with the tracheal intubation of Example 1 of the present disclosure;

FIG. 3 is a schematic structural diagram of a sealing device for endoscope intubation of Example 1 of the present disclosure;

FIG. 4 is a schematic structural diagram of a sealing device for endoscope intubation of Example 2 of the present disclosure;

FIG. 5 is a schematic structural diagram of a sealing device for endoscope intubation of Example 2 of the present disclosure along the axis in a front view;

FIG. 6 is a schematic structural diagram of a sealing device for endoscope intubation of Example 3 of the present disclosure; and

FIG. 7 is a schematic structural diagram of a sealing device for endoscope intubation of Example 3 of the present disclosure along the axis in a front view.

Reference numerals: 1. main body part; 10. axis; 11. through hole; 12. gap; 13. distal end; 14. proximal end; 2. sealing part; 20. first guide surface; 3. friction part; 30. first surface; 31. second guide surface; 4. operating handle; 5. catheter; 6. tracheal intubation.

### DETAILED DESCRIPTION OF THE EMBODIMENTS

A clear and complete description of the technical solutions of the present disclosure will be given below in connection with the drawings. Obviously, the described embodiments are a portion of the embodiments of the present disclosure and not all of the embodiments. Based on the embodiments of the present disclosure, all other embodiments obtained by those skilled in the art without inventive effort shall fall within the protection scope of the present disclosure.

In the description of the present disclosure, it should be understood that the terms “thickness”, “up”, “down”, “top”, “bottom”, “inside” and “outside” and the like indicate orientations or positional relationships based on those shown in the accompanying drawings, and are intended only for the purpose of facilitating the description of the present disclosure and to simplify the description and are not indicative of, or suggestive of, that the referred device or element must have a particular orientation, be constructed and operated with a particular orientation, and therefore are not to be understood as limitations of the present disclosure. Furthermore, the terms “first” and “second” are used for descriptive purposes only and are not to be understood as indicating or implying relative importance or implicitly indicating the number of indicated technical features. Therefore, the features defined as “first” and “second” can include one or more features explicitly or implicitly. In the description of the present disclosure, the meaning of “a plurality” is two or more, and “at least one” can be one, two or more, unless otherwise specifically defined.

The applicant noticed that the minimally invasive technology based on electronic endoscope clinically is widely used in many hospital departments such as respiratory department, digestive department, urology department and otolaryngology department, in which the respiratory intensive care unit conducts tracheal intubation under visual monitoring to guide the intubation to the appropriate position. Ventilation and oxygen supply are sometimes needed in the operation process, which requires that the joint of the catheter of the tracheoscope and the tracheal intubation has a certain sealing performance to avoid air leakage. At the same time, the tracheal intubation needs to move with the tracheoscope during the operation process, and be performed in a visual state during the whole operation to avoid damaging the airway.

In the prior art, the flexible cone piece is sleeved over the catheter to be in interference fit and inserted with the tracheal intubation, which has poor adaptability to different sizes of tracheal intubation. When the flexible cone piece is inserted with the tracheal intubation too tightly, it is easy to pull the tracheal intubation to move when withdrawing the tracheal catheter, resulting in damage to the airway; and when the flexible cone piece is inserted with the tracheal intubation too loosely, it is easy to result in poor sealing performance and air leakage during oxygen delivery by the tracheal intubation.

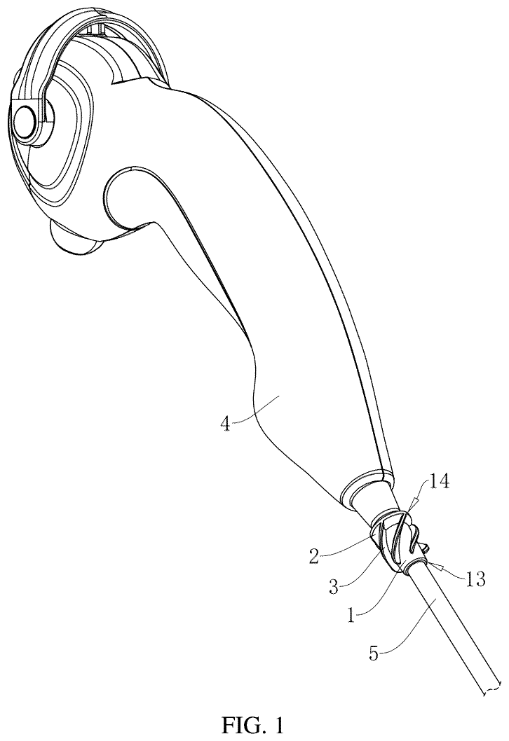

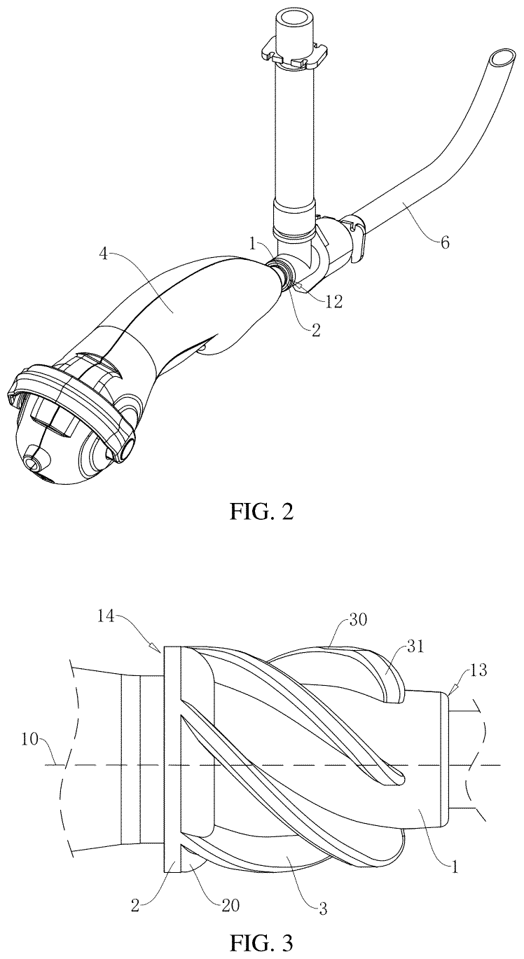

In view of this, with reference to FIG. 1 to FIG. 7, an embodiment of the present disclosure provides a sealing device for endoscope intubation, aiming at solving at least one of the above technical problems.

### Example 1

Referring to FIG. 1 to FIG. 3, a sealing device for endoscope intubation, configured for connecting hermetically the catheter 5 of a tracheoscope and the tracheal intubation 6, includes a main body part 1, a sealing part 2 and a friction part 3, wherein the main body part 1 has an axis 10, the main body part 1 is configured for connecting to the catheter 5, at least a portion of the main body part 1 is configured to be able to be inserted into the tracheal intubation 6, and there is a gap 12 between the main body part 1 and the inner tube wall of the tracheal intubation 6; the sealing part 2 is annularly arranged on the main body part 1, and the sealing part 2 is configured to be able to seal the gap 12; and the friction part 3 is arranged on the main body part 1, and at least portion of the friction part 3 is configured to abut against the inner wall of the tracheal intubation 6.

It should be noted that in this example, the main body part 1 is provided with a through hole 11 along the axis 10 passing therethrough so as to be sleeved over the catheter 5. Corresponding to the shape of the inner wall of the tracheal intubation 6, in this example, the sealing part 2 is in an annular structure surrounded the main body part 1 around the axis 10, and the sealing connection of the catheter 5 and the tracheal intubation 6 is realized by the main body part 1 and the sealing part 2, to avoid leakage when oxygen is delivered by the tracheal intubation 6, while the friction part 3 abuts against the tracheal intubation 6 in the gap 12 to form a certain friction force, so as to maintain the synchronous mobility of the catheter 5 and the tracheal intubation 6.

Specifically, in some embodiments, referring to FIG. 1 to FIG. 3, the friction part 3 is configured to generate an elastic deformation when the main body part 1 is inserted into the tracheal intubation 6, to abut against the inner wall of the tracheal intubation 6. That is, in this example, the friction part 3 is made of elastic material, and the inner wall of the tracheal intubation 6 presses the friction part 3 with respect to the main body part 1 during the insertion of the main body part 1 into the tracheal intubation 6, thereby forming an interference fit between the friction part 3 and the tracheal intubation 6, and reducing the possibility of the tracheal intubation 6 loosening when the catheter 5 moves.

In order to further improve the synchronous mobility between the tracheal intubation 6 and the catheter 5, referring to FIG. 1 to FIG. 3, in this example, a plurality of friction parts 3 are provided, and the plurality of the friction parts 3 are arranged at intervals in a circumferential direction around the axis 10, and the adjacent friction parts 3 are evenly spaced. Furthermore, the friction part 3 extends spirally around the axis 10.

It can be understood that in this example, the plurality of friction parts 3 are designed as blade shape and distributed on the surface of the main body part 1, so as to realize uniform abutting against the tracheal intubation 6 during the insertion of the main body part 1 into the tracheal intubation 6, and improve the stability of connecting the tracheal intubation 6. At the same time, the elastic and flaky friction parts 3 can better adapt to tracheal intubation 6 with different tube diameters compared with the solid cone, so as to improve the compatibility of sealing fit.

In some embodiments, referring to FIG. 1 to FIG. 3, the main body part 1 has a distal end 13 and a proximal end 14 in the extending direction of the axis 10. It can be understood that in this example, taking FIG. 1 as an example, the above distal end 13 refers to the end far away from the operating handle 4 held by a person, and the proximal end 14 refers to the end close to the operating handle 4 held by a person.

Referring to FIG. 1 to FIG. 3, the sealing part 2 is provided with a first guide surface 20, the first guide surface 20 is arranged towards the distal end 13, and the first guide surface 20 is configured for guiding the sealing part 2 to be embedded into the gap 12. That is, the first guide surface 20 is set as an arc surface in this example, which can guide the sealing part 2 to align with the tracheal intubation 6 and reduce the resistance in an insertion process when the sealing part 2 is inserted into the tracheal intubation 6.

Similarly, referring to FIG. 3, the friction part 3 is provided with a second guide surface 31, the second guide surface 31 is arranged towards the distal end 13, and the second guide surface 31 is configured for guiding the friction part 3 to be embedded into the gap 12. That is, the second guide surface 31 is also set as an arc surface in this example, which can guide the friction part 3 to align with the tracheal intubation 6 and reduce the resistance in the insertion process when the friction part 3 is inserted into the tracheal intubation 6.

It should be noted that in this example, the main body part 1, the sealing part 2 and the friction part 3 are integrally formed, that is, the main body part 1, the sealing part 2 and the friction part 3 can all be integrally molded with elastic materials such as elastic silica gel.

In addition, in order to improve the overall structural stability of the sealing part 2 and a plurality of friction parts 3, referring to FIG. 1 to FIG. 3, the friction parts 3 are connected with the sealing part 2, and in this example, each friction part 3 is integrally arranged on the side of the sealing part 2 near the distal end 13. Through the above design, in the process of inserting the main body part 1 into the tracheal intubation 6, a stable friction force is formed between the friction part 3 and the tracheal intubation 6 in advance, and finally the gap 12 is sealed by the sealing part 2 to form a stable connection.

The example of the present disclosure further discloses an endoscope, including the above-mentioned sealing device for endoscope intubation, and it can be understood that the endoscope can have all the technical features and corresponding beneficial effects of the above-mentioned sealing device for endoscope intubation, which will not be described in detail here.

In some embodiments, referring to FIG. 1 to FIG. 3, the endoscope further includes an operating handle 4 and a catheter 5 connected with the operating handle 4, wherein the sealing device for endoscope intubation is sleeved over the catheter 5, and the sealing device for endoscope intubation is fixedly connected with the operating handle 4.

It should be noted that in this example, the operating handle 4 and the catheter 5 are in sealing fit through the elastic sealing device for endoscope intubation with the tracheal intubation 6, which is beneficial to realize the buffering transition from the harder operating handle 4 to the softer catheter 5 and the tracheal intubation 6, and to reduce the problem of the joint of the catheter 5 and the tracheal intubation 6 being folded when the tracheal intubation 6 is sharply bent.

### Example 2

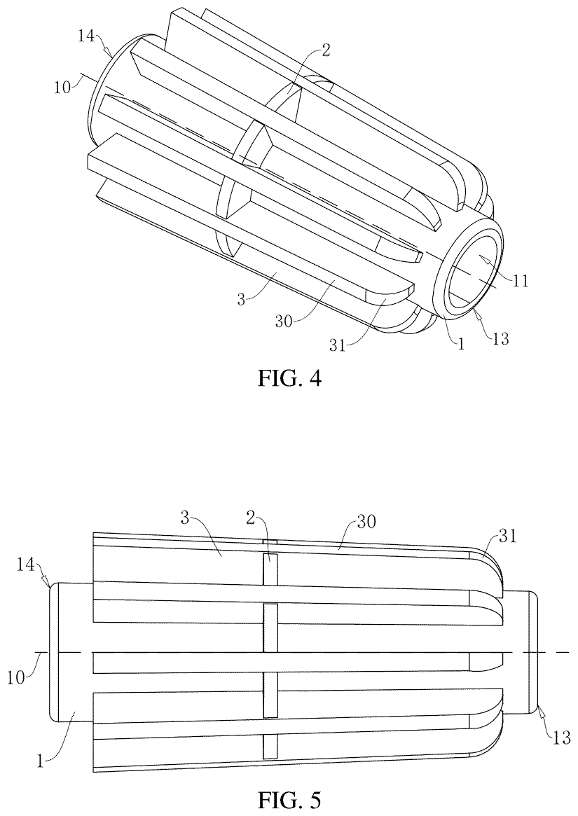

Referring to FIG. 3 to FIG. 4, the difference between this example and Example 1 is that the friction part 3 is set as a straight blade structure parallel to the extension direction of the axis 10, the sealing part 2 is set in the middle of the friction part 3 in its own extension direction, and the friction part 3 penetrates through the sealing part 2.

It should be noted that, referring to FIG. 4, the friction part 3 has a first surface 30 facing away from the main body part 1, and the vertical distance between at least part of the first surface 30 and the axis 10 gradually increases from the distal end 13 to the proximal end 14. In this example, taking the gradually increased vertical distance between the first surface 30 and the axis 10 for example, the friction parts 3 are evenly circumferentially set around the axis 10, the outer contour formed by each first surface 30 around the axis 10 is actually of a circular truncated cone-shaped structure, that is, as the main body part 1 is gradually inserted into the tracheal intubation 6, the extrusion force of the tracheal intubation 6 on the friction parts 3 gradually increases, and at the same time, the vertical distance between the first surface 30 of the friction part 3 near the distal end 13 and the axis 10 can be set to be less than or equal to the inner diameter of the tracheal intubation 6. Under this circumstances, it is less difficult for the friction part 3 to be embedded in the tracheal intubation 6, and the first surface 30 on the proximal end 14 can also ensure that the sufficient friction is formed between the friction portion 3 and the tracheal intubation 6.

In the above settings, referring to FIG. 3 and FIG. 4, the following methods can be adopted.

First, the maximum size of the main body part 1 in the direction perpendicular to the axis 10 gradually increases from the distal end 13 to the proximal end 14, that is, the main body part 1 is set to as a circular truncated cone-like structure, wherein the size of the outer circle of the section of the portion of the main body part 1 near the distal end 13 perpendicular to the axis 10 is set smaller than the size of the outer circle of the section of the portion of the main body part 1 near the proximal end 14 perpendicular to the axis 10. On this basis, in some embodiments, if the friction parts 3 have the same height, that is, the distance between the first surface 30 and the outer surface of the main body part 1 is the same from the distal end 13 to the proximal end 14, it needs to conform to the inclined outer surface of the main body part 1 in the extension direction of the axis 10 when it is arranged in the main body part 1. In addition, if the distance between the first surface 30 and the outer surface of the main body part 1 gradually increases from the distal end 13 to the proximal end 14, the above requirements can also be met.

Second, the distance between the first surface 30 and the outer surface of the main body part 1 gradually increases from the distal end 13 to the proximal end 14, that is, the friction part 3 is designed as a blade structure with a gradually changed width in the direction perpendicular to the axis 10. In some embodiments, if the main body part 1 is of a straight tube structure, that is, the largest dimension of the main body part 1 in the direction perpendicular to the axis 10 is the same from the distal end 13 to the proximal end 14, and it is also possible to realize that the outer contour formed by each first surface 30 around the axis 10 is of a circular truncated cone-shaped structure.

The above design makes it easier for the main body part 1 and the friction part 3 to be aligned and inserted smoothly during inserting in the tracheal intubation 6, and can flexibly adapt to tracheal intubations 6 with more tube diameter specifications, that is, increase or decrease the size inserted into the tracheal intubation 6 as required to form appropriate friction. Both of the above two methods are adopted at the same time in this example, and may be alternatively provided in other examples.

### Example 3

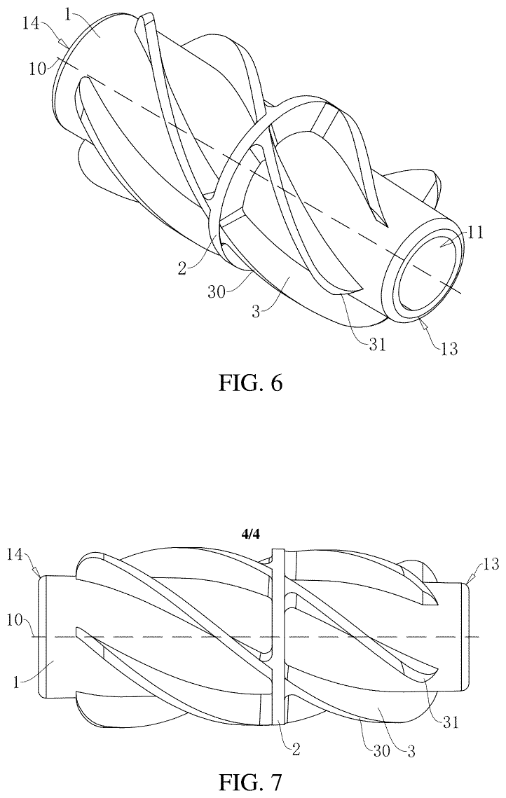

Referring to FIG. 6 and FIG. 7, the difference between this example and Example 1 is that although the friction part 3 extends spirally around the axis 10, the sealing part 2 is arranged in the middle of the friction part 3 in its own extension direction, and the friction part 3 penetrates through the sealing part 2. It should be noted herein that, in the above-described arrangement, another reason why at least part of the friction part 3 is arranged on the side of the sealing part 2 near the distal end 13 is that: in order to reduce the resistance of the main body part 1 and the friction part 3 in the initial stage of inserting in the tracheal intubation 6, the perpendicular distance between the first surface 30 of the friction part 3 near the distal end 13 and the axis 10 is smaller relative to that near the proximal end 14, and the sealing part 2 needs to adapt to the gradually changed size of the friction part 3, that is, the size of the sealing part 2 is set to be consistent with the size of the friction part 3 at the position. If the sealing part 2 is set too close to the distal end 13, the dimensions of the friction part 3 actually result in a smaller amount of extrusion deformation during inserting in the tracheal intubation 6, and it is also difficult for the sealing part 2 to achieve a good sealing effect. Therefore, the sealing part 2 is set in the middle of the friction part 3 in the extension direction of the axis 10 or on the side far from the distal end 13.

In addition, in other examples, a plurality of sealing parts 2 may be provided (not shown in the figure), and the plurality of sealing parts 2 are arranged along the extending direction of the axis 10. The sealing parts 2 also have the function of forming friction with the tracheal intubation 6. Increasing the number of sealing parts 2 can not only improve the connection sealing performance between the catheter 5 and the tracheal intubation 6, i.e., achieving the multiple sealing effect, but also increase the connection stability between the catheter 5 and the tracheal intubation 6, i.e., further increasing the frictional contact area.

The above describes in detail a sealing device for endoscope intubation and an endoscope provided by embodiments of the present disclosure, and applies specific examples to illustrate the principles and implementations of the present disclosure. the above embodiments are only used to illustrate the technical solutions and the core ideas of the present disclosure, a person of skill in the art should understand that it is still possible to make modifications to the technical solutions documented in the foregoing embodiments, or to make equivalent substitutions for some or all of the technical features therein; and these modifications or substitutions do not take the essence of the corresponding technical solutions out of the scope of the technical solutions of the embodiments of the present disclosure.

1. A sealing device for endoscope intubation, configured for sealing connection of a catheter of a tracheoscope and a tracheal intubation, and comprising:
a main body part, wherein the main body part has an axis, the main body part is configured for connecting to the catheter, at least a portion of the main body part is configured to be able to be inserted into the tracheal intubation, and a gap exists between the main body part and an inner tube wall of the tracheal intubation;
a sealing part, wherein the sealing part is annularly arranged on the main body part, and the sealing part is configured to be able to seal the gap; and
a friction part, wherein the friction part is arranged on the main body part, and at least portion of the friction part is configured to abut against an inner wall of the tracheal intubation.

2. The sealing device for endoscope intubation according to claim 1, wherein
the friction part is configured to generate an elastic deformation when the main body part is inserted into the tracheal intubation, to abut against the inner wall of the tracheal intubation.

3. The sealing device for endoscope intubation according to claim 1, wherein
a plurality of friction parts are provided, and the plurality of friction parts are arranged at intervals in a circumferential direction around the axis.

4. The sealing device for endoscope intubation according to claim 2, wherein
an extending direction of the friction part intersects or is parallel to an extending direction of the axis.

5. The sealing device for endoscope intubation according to claim 2, wherein
the friction part extends spirally around the axis.

6. The sealing device for endoscope intubation according to claim 1, wherein
the main body part has a distal end and a proximal end in an extending direction of the axis; and
the friction part has a first surface facing away from the main body part, and a vertical distance between at least part of the first surface and the axis gradually increases from the distal end to the proximal end.

7. The sealing device for endoscope intubation according to claim 6, wherein
a maximum dimension of the main body part in a direction perpendicular to the axis gradually increases from the distal end to the proximal end; and
a distance between the first surface and an outer surface of the main body part gradually increases or is the same from the distal end to the proximal end.

8. The sealing device for endoscope intubation according to claim 6, wherein
a maximum dimension of the main body part in a direction perpendicular to the axis is the same from the distal end to the proximal end; and
a distance between the first surface and an outer surface of the main body part gradually increases from the distal end to the proximal end.

9. The sealing device for endoscope intubation according to claim 1, wherein
the main body part has a distal end and a proximal end in an extending direction of the axis; and
the sealing part is provided with a first guide surface, the first guide surface is arranged towards the distal end, and the first guide surface is configured for guiding the sealing part to be embedded into the gap.

10. The sealing device for endoscope intubation according to claim 1, wherein
the main body part has a distal end and a proximal end in an extending direction of the axis; and
the friction part is provided with a second guide surface, the second guide surface is arranged towards the distal end and the second guide surface is configured for guiding the friction part to be embedded into the gap.

11. The sealing device for endoscope intubation according to claim 1, wherein
a plurality of sealing parts are provided, and the plurality of sealing parts are arranged along an extending direction of the axis.

12. The sealing device for endoscope intubation according to claim 1, wherein
the friction part is connected with the sealing part; and/or
the main body part has a distal end and a proximal end in an extending direction of the axis, and at least portion of the friction part is arranged at one side of the sealing part close to the distal end.

13. The sealing device for endoscope intubation according to claim 1, wherein
the main body part, the sealing part and the friction part are integrally formed.

14. An endoscope, comprising the sealing device for endoscope intubation according to claim 1.

15. The endoscope according to claim 14, wherein the endoscope further comprises:
an operating handle and a catheter connected with the operating handle, wherein the sealing device for endoscope intubation is sleeved over the catheter, and the sealing device for endoscope intubation is fixedly connected with the operating handle.

16. The endoscope according to claim 14, wherein
the friction part is configured to generate an elastic deformation when the main body part is inserted into the tracheal intubation, to abut against the inner wall of the tracheal intubation.

17. The endoscope according to claim 14, wherein
a plurality of friction parts are provided, and the plurality of friction parts are arranged at intervals in a circumferential direction around the axis.

18. The endoscope according to claim 16, wherein
an extending direction of the friction part intersects or is parallel to an extending direction of the axis.

19. The endoscope according to claim 16, wherein
the friction part extends spirally around the axis.

20. The endoscope according to claim 14, wherein
the main body part has a distal end and a proximal end in an extending direction of the axis; and
the friction part has a first surface facing away from the main body part, and a vertical distance between at least part of the first surface and the axis gradually increases from the distal end to the proximal end.
